(19) United States
(12) Patent Application Publication
(10) Pub. No.: US 2014/0067049 A1
(43) Pub. Date: Mar. 6, 2014

(54) Integrated Dilation Balloon and Valve Prosthesis Delivery System

(71) Applicant: Declan Costello, Ballybrit (IE)

(72) Inventor: Declan Costello, Ballybrit (IE)

(73) Assignee: Medtronic Vascular Galway Limited, Ballybrit (IE)

(21) Appl. No.: 13/604,098

(22) Filed: Sep. 5, 2012

(51) Int. Cl. A61F 2/24

(52) U.S. Cl. 623/2.11

(57) ABSTRACT

An integrated balloon dilation and valve prosthesis delivery device is provided having a capsule that is configured to surround a collapsed valve prosthesis and that contains a balloon on an exterior surface.

### BACKGROUND OF THE INVENTION

1. Field of the Invention

The present invention is related to an integrated dilation balloon and prosthetic heart valve delivery system.

2. Background Art

Cardiac valves exhibit two types of pathologies: regurgitation and stenosis. Regurgitation is the more common of the two defects. Either defect can be treated by a surgical repair. In addition, stenosis can be treated through balloon dilation, also known as valvuloplasty, by placing a balloon catheter inside the valve and inflating the balloon in an effort to increase the opening size of the valve and thus improve blood flow.

Under certain conditions, the cardiac valve must be replaced. Standard approaches to valve replacement require cutting open the patient's chest and heart to access the native valve. Such procedures are traumatic to the patient, require a long recovery time, and can result in life threatening complications. Therefore, many patients requiring cardiac valve replacement are deemed to pose too high a risk for open heart surgery due to age, health, or a variety of other factors. These patient risks associated with heart valve replacement are lessened by the emerging techniques for minimally invasive valve repair, but still many of those techniques require arresting the heart and passing the blood through a heart-lung machine.

Efforts have been focused on percutaneous transluminal delivery of replacement cardiac valves to solve the problems presented by traditional open heart surgery and minimally-invasive surgical methods. In such methods, a valve prosthesis is compacted for delivery in a catheter and then advanced, for example, through an opening in the femoral artery and through the descending aorta to the heart, where the prosthesis is then deployed in the aortic valve annulus. Often in the case of a stenosed valve, valvuloplasty is performed prior to delivery of the valve prosthesis. In addition, after deployment of the valve prosthesis, balloon dilation can be performed to post dilate the valve prosthesis and ensure that the valve prosthesis is adequately seated in the native valve annulus.

### BRIEF SUMMARY OF THE INVENTION

Provided herein is a valve prostheses delivery system that generally includes a delivery system having a capsule at a distal end. The capsule surrounds a compressed valve prosthesis and a balloon is provided on an exterior surface of the capsule. Such configurations achieve numerous goals. For example, such a configuration allows for a reduction in the number of devices used to treat a stenosed valve through balloon dilation and to deliver a valve prosthesis.

In view thereof, disclosed herein are aspects of an integrated balloon dilation and valve prosthesis delivery system which is generally designed to include an inner shaft assembly including an intermediate portion providing a coupling structure configured to selectively engage a prosthetic valve, and an outer shaft assembly including a delivery sheath capsule, and expandable balloon attached to an exterior surface of the capsule, and an inflation lumen extending along the length of the outer shaft assembly.

In another exemplary embodiment, disclosed herein are aspects of a method of treating a valve disorder in a patient's heart which generally includes delivering an integrated balloon dilation and valve prosthesis delivery device through an anatomic passageway into a native valve within a heart, expanding a balloon on the integrated device to contact and dilate the native valve, and proximately retracting a capsule on the integrated device relative to a valve prosthesis to deploy the valve prosthesis in the native valve.

### BRIEF DESCRIPTION OF THE DRAWINGS/FIGURES

The accompanying figures, which are incorporated herein, form part of the specification and illustrate embodiments of a valve prosthesis. Together with the description, the figures further serve to explain the principles of and to enable a person skilled in the relevant art(s) to make, use, and implant the valve prosthesis described herein. In the drawings, like reference numbers indicate identical or functionally similar elements.

FIG. 1 is a sectional view of a valve prosthesis delivery system and integrated dilation balloon according to an aspect of this disclosure.

FIG. 2 is a side view of a valve prosthesis delivery system and integrated dilation balloon according to an aspect of this disclosure.

FIG. 3 is a schematic view of a stenosed aortic valve and a guide wire for a valve prosthesis delivery system and integrated dilation balloon according to an aspect of this disclosure.

FIG. 4 is a schematic view of a stenosed aortic valve and a valve prosthesis delivery system and integrated dilation balloon according to an aspect of this disclosure.

FIG. 5 is a schematic view of a stenosed aortic valve and a valve prosthesis delivery system and integrated dilation balloon according to an aspect of this disclosure.

FIG. 6 is a schematic view of a stenosed aortic valve and a valve prosthesis delivery system and integrated dilation balloon according to an aspect of this disclosure.

FIG. 7 is a schematic view of a valve prosthesis and valve prosthesis delivery system and integrated dilation balloon according to an aspect of this disclosure.

FIG. 8 is a schematic view of a valve prosthesis and valve prosthesis delivery system and integrated dilation balloon according to an aspect of this disclosure.

FIG. 9 is a schematic view of a valve prosthesis and valve prosthesis delivery system and integrated dilation balloon according to an aspect of this disclosure.

FIG. 10 is a schematic view of a valve prosthesis after deployment according to an aspect of this disclosure.

FIG. 11 is a sectional view of a valve prosthesis delivery system and integrated dilation balloon according to an aspect of this disclosure.

FIG. 12 is a side view of a valve prosthesis delivery system and integrated dilation balloon according to an aspect of this disclosure.

### DETAILED DESCRIPTION OF THE INVENTION

The following detailed description of a valve prosthesis delivery system refers to the accompanying figures that illustrate exemplary embodiments. Other embodiments are possible. Modifications can be made to the embodiments described herein without departing from the spirit and scope of the present invention. Therefore, the following detailed description is not meant to be limiting.

The present invention is directed to a heart valve prosthesis delivery system including a balloon integrated onto an exterior surface of the delivery system capsule. The integrated delivery system is a single device that allows a practitioner to perform balloon dilation on native valve leaflets and to deliver a valve prosthesis percutaneously to the heart to replace the function of a native valve. For example, the valve prosthesis can replace a bicuspid or a tricuspid valve such as the aortic, mitral, pulmonary, or tricuspid heart valve.

Typically, balloon dilation, also known as valvuloplasty, is performed using a device separate from the valve prosthesis delivery system. The practitioner first percutaneously inserts the balloon dilation device into the patient, expands the dilation balloon against a native stenosed valve to dilate the valve, deflates the dilation balloon and then removes the balloon dilation device from the patient. At this point, the practitioner can percutaneously insert the valve prosthesis delivery system into the patient to deliver and deploy the valve prosthesis. Occasionally, after the valve prosthesis is deployed, post dilation with the balloon dilation device is required in order to adequately seat the valve prosthesis in the native valve annulus, to prevent valve prosthesis leakage, and/or to remove residual calcification. In this case, the balloon dilation device must be reinserted into the patient after removal of the valve prosthesis delivery system.

An introducer is typically used for a procedure involving balloon dilation and valve prosthesis delivery. The introducer allows for the exchange of the balloon dilation device and valve prosthesis delivery system into and out of the patient. However, the introducer also increases the total size and profile that is inserted into the patient. The profile of a device is the total diameter that must be passed into the patient's vasculature.

Valve prostheses typically have eyelets to attach the valve prostheses to a delivery system. The eyelets attach to tabs which retain the valve prosthesis. In addition, valve prosthesis delivery systems typically include an outer sheath or capsule that surrounds the collapsed valve prosthesis during delivery to the implantation site. During deployment, the capsule is withdrawn over the valve prosthesis.

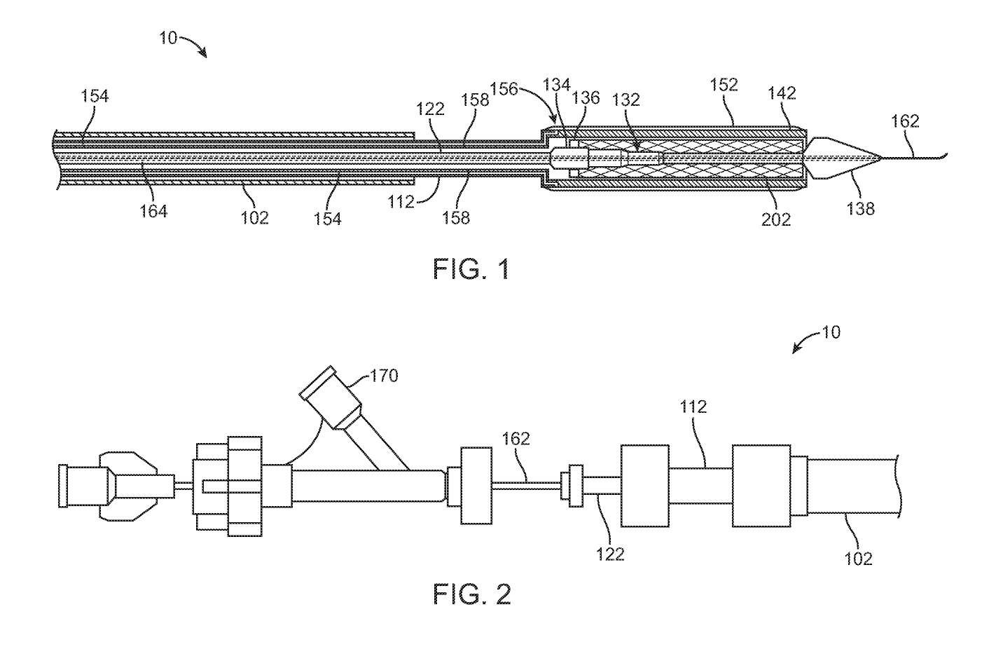

Referring now to FIGS. 1-2 the integrated delivery system for valve prosthesis 202 includes integrated delivery system 10 that includes an outer sheath 102, a pusher tube 112, and a central tube 122, each of which can be concentrically aligned and permit relative motion with respect to each other. At a distal end of pusher tube 112 is a capsule 142. Pusher tube 112 also includes an inflation lumen 154. In one aspect of the invention, inflation lumen 154 extends along the length of pusher tube 112 and is defined by a space between the pusher tube 112 and a wall 158. In one aspect, inflation lumen 154 is an annular inflation lumen defined by the annular space between wall 158 and pusher tube 112. In a further aspect, wall 158 is located within pusher tube 112 such that inflation lumen 154 extends along the interior of pusher tube 112. In an alternate aspect, wall 158 can surround pusher tube 112 such that inflation lumen 154 extends long the exterior of pusher tube 112. In an alternate aspect of the invention, integrated delivery system 10 can include a non-annular inflation lumen and can include one or more single point inflation lumens that extend along the length of pusher tube 112. The one or more single point inflation lumens can extend along the interior of pusher tube 112 or along the exterior of pusher tube 112.

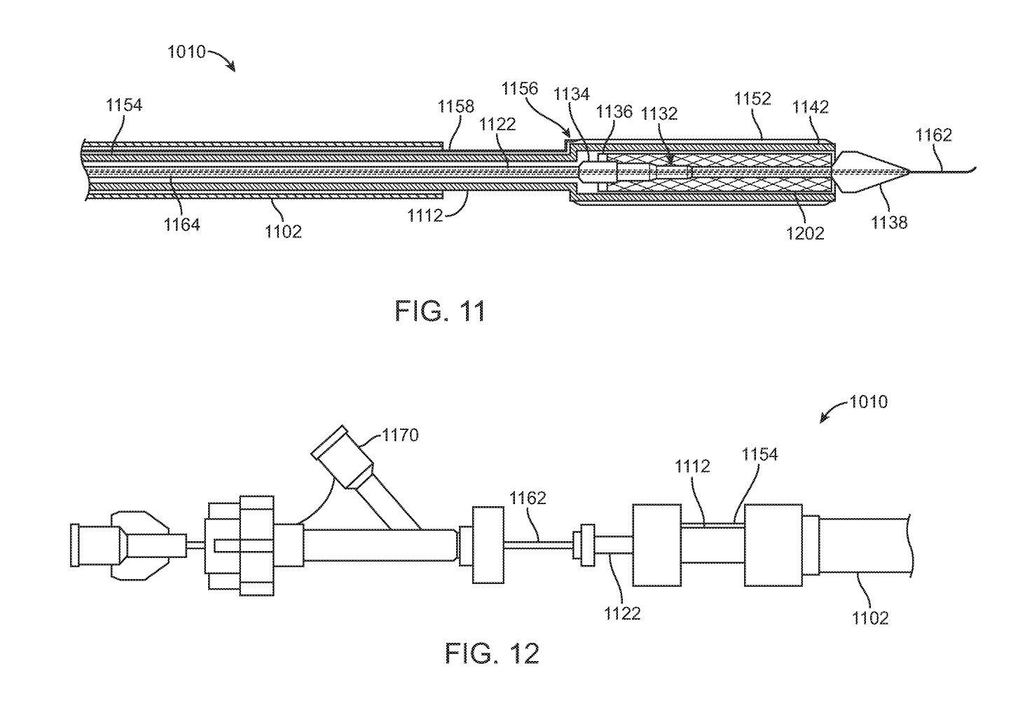

In a further aspect of the invention, shown in FIGS. 11-12, inflation lumen 1154 is a single point lumen that extends along the exterior of pusher tube 1112 into the proximal end of balloon 1152. Inflation lumen 1154 is defined by a space located between pusher tube 1112 and wall 1158. In this aspect, the radial width of inflation lumen 1154 is small enough not to increase the overall diameter and profile of delivery device 1010.

Central tube 122 includes guide wire lumen 164 which passes over guide wire 162. Ata distal end of central tube 122 is plunger assembly 132. Capsule 142 surrounds plunger assembly 132 and collapsed valve prosthesis 202 and restrains valve prosthesis 202 in the radial direction during delivery of valve prosthesis 202. Plunger assembly 132 includes hub 134 at a proximal end and tip 138 at a distal end. Tip 138 facilitates the advancement of integrated delivery system 10 through the patient's vasculature. Hub 134 includes one or more tabs 136 for retaining valve prosthesis 202 on plunger assembly 132. Tabs 136 also prevent the pre-release of valve prosthesis 202 and assist in retaining valve prosthesis 202 during recapture. The top surface of tabs 136 interact with the inner surface of capsule 142 to form an interference fit.

Balloon 152 is attached to the exterior surface of capsule 142. Balloon 152 is sealed at the distal end of capsule 142, adjacent tip 138. Balloon 152 is also sealed to the proximal end of capsule 142. At the proximal end, balloon 152 is connected to inflation lumen 154 through opening 156. In one aspect, opening 156 extends through capsule 142. In an alternate aspect of the invention shown in FIGS. 11-12, opening 1156 is positioned on an exterior of capsule 1142. Opening 156 is located proximal to hub 134 and valve prosthesis 202 on integrated delivery system 10. Because the collapsed valve prosthesis abuts the interior of capsule 142, providing opening 156 at this location allows for integration of an inflation lumen and dilation balloon onto the delivery device without increasing the overall profile and size of the delivery device. In addition, capsule 142 must be sufficiently rigid to resist the radially outward forces exerted by compressed valve prosthesis 202 and the radially inward forces exerted by expanded balloon 152. Inflation port 170 is connected to inflation lumen 154 and is provided to transmit inflation fluid into balloon 152 to expand balloon 152. In the collapsed state, the width of balloon 152 is marginal so as not to increase the overall size and profile of integrated delivery system 10. Balloon 152 can be manufactured by a person skilled in the art and can utilize common materials including but not limited to as Pebax, Grilimid, nylon in various grades, and latex. In one aspect, balloon 152 is a double wall balloon. The double wall thickness of balloon 152 can range from approximately 0.001 inches to approximately 0.005 inches and will be dictated by material and inflation pressure.

In one aspect of the invention, valve prosthesis 202 is self-expandable. In an alternate aspect of the invention, valve prosthesis can be balloon expandable. An integrated delivery system for a balloon expandable valve would also include a second inflation lumen connected to a second balloon. The second balloon would be placed inside collapsed valve prosthesis 202 in order to expand valve prosthesis 202 in a native valve annulus.

Integrating balloon 152 onto capsule 142 allows for the balloon dilation procedure and the valve delivery procedure to be performed using a single device. In addition, post dilation of the valve prosthesis and native valve can be performed with the same integrated delivery device. Because both procedures can be performed with a single device, devices no longer must be exchanged into and out of the body. Therefore, with the integrated delivery system an introducer is no longer necessary thus decreasing the overall device profile that must be inserted into the body to perform the procedures. Reducing the overall profile allows for a smaller insertion hole into the body which leads to a reduction in vessel closure complications. In addition, reducing the number of devices used in the valve repair procedure also decreases the total procedure time. A typical balloon dilation and valve implantation procedure typically requires approximately 20 to approximately 30 minutes of procedure time. Integrating the balloon dilation device into the valve delivery device could save approximately 5 to approximately 10 minutes of total procedure time because a practitioner does not need to exchange a different balloon dilation device and valve prosthesis delivery device. Thus, a patient undergoing the procedure has less time on anesthesia and also has less risk of bleeding. Integrating the balloon dilation device into the valve prosthesis delivery system is beneficial for any access method, including transfemoral, transeptal, transapical, transradial, transsubclavian, or transatrial.

In addition, it is advantageous to place the balloon on the exterior surface of the capsule, rather than adjacent the capsule. The size of balloon 152 is small enough such that the addition of balloon 152 onto the exterior of capsule 142 will not increase the overall profile of delivery device. If the dilation balloon is placed adjacent to capsule, the device must be advanced into the ventricle and could cause damage. In using such a device, there is a risk of ventricular perforation resulting in damage to the heart.

In one aspect of the invention, FIG. 10 shows valve prosthesis 202 in the expanded configuration. In the expanded configuration, valve prosthesis 202 is disengaged from tabs 136.

The valve prosthesis can replace the function of a tricuspid or bicuspid heart valve including the mitral valve, the aortic valve, the pulmonary valve, or the tricuspid valve. The valve can be delivered, for example, transfemorally, transeptally, transapically, transradially, transsubclavian, or transatrially.

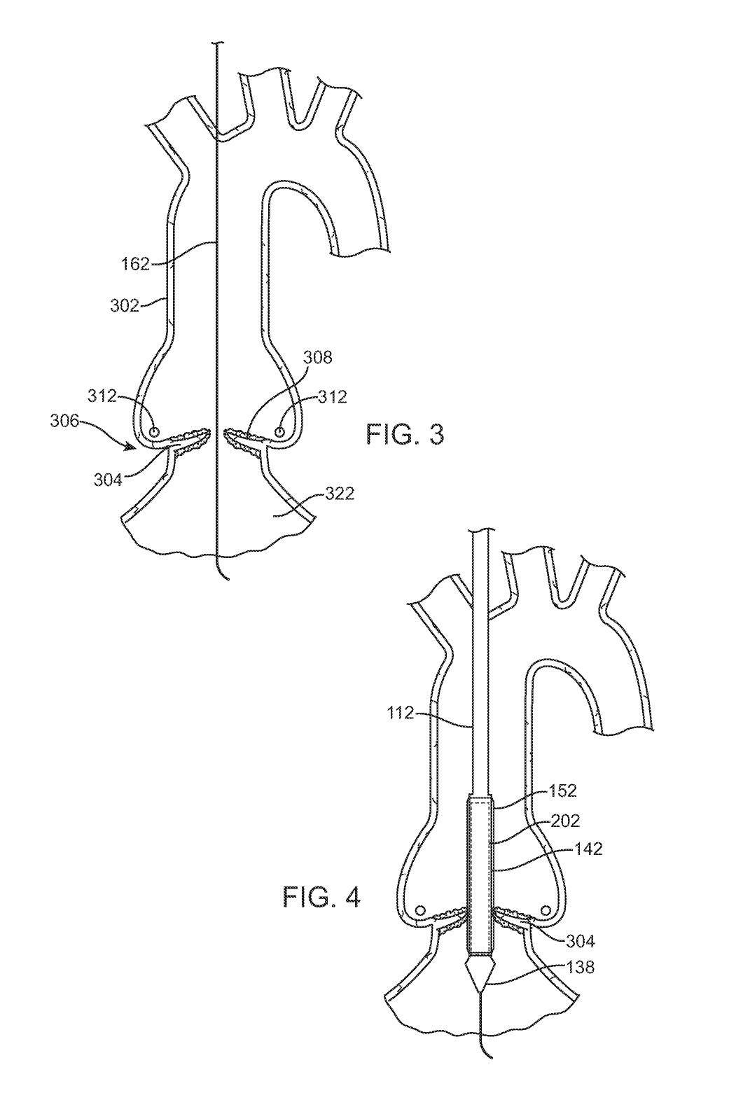

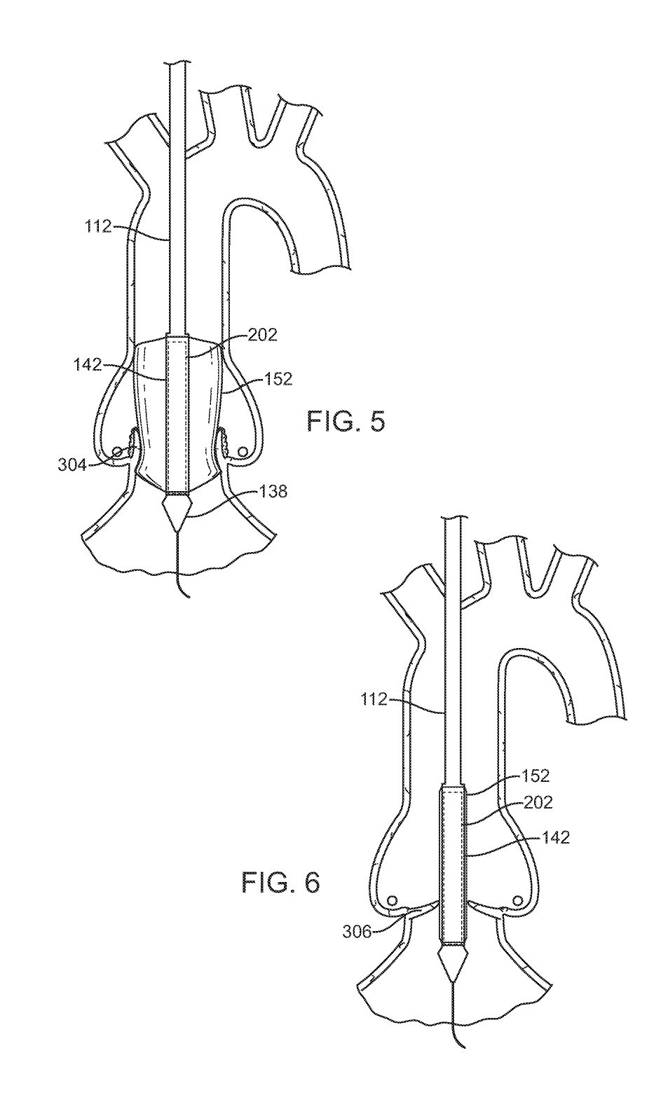

Balloon dilation and implantation of the valve prosthesis will now be described with respect to FIGS. 3-10. As discussed above, in one aspect of the invention the valve prosthesis comprises a self-expanding frame that can be compressed to a contracted delivery configuration onto hub 134 on plunger assembly 132. In an alternative aspect of the invention, the valve prosthesis frame can be balloon expandable. The self-expanding frame design requires a loading system to crimp valve prosthesis 202 to the delivery size, while allowing the proximal end of valve prosthesis 202 to protrude from the loading system so that the proximal end can be attached to tabs 136.

The valve prosthesis and plunger assembly can then be loaded into capsule 142. In the transfemoral approach, the integrated delivery system and valve prosthesis are advanced into the patient's descending aorta. The integrated delivery system then is advanced, under fluoroscopic guidance, over the aortic arch, through the ascending aorta 302 and into the aortic annulus 306, mid-way across aortic valve 304. In the trassubclavian approach, the integrated delivery system and valve prosthesis are advanced through the subclavian artery into the ascending aorta 302 and into the aortic annulus 306, mid-way across the aortic valve 304.

Once positioning of the integrated delivery system in the aortic annulus 306 is confirmed, balloon dilation can be performed by inflating balloon 152 into the native valve leaflets to dilate aortic valve 304 and to treat calcium buildup 308 by deforming the valve leaflets against the aortic wall adjacent aortic valve 304. Balloon 152 is expanded by passing fluid through inflation lumen 154 into balloon 152. After balloon dilation is performed, the fluid is removed deflating balloon 152, as shown in FIG. 6.

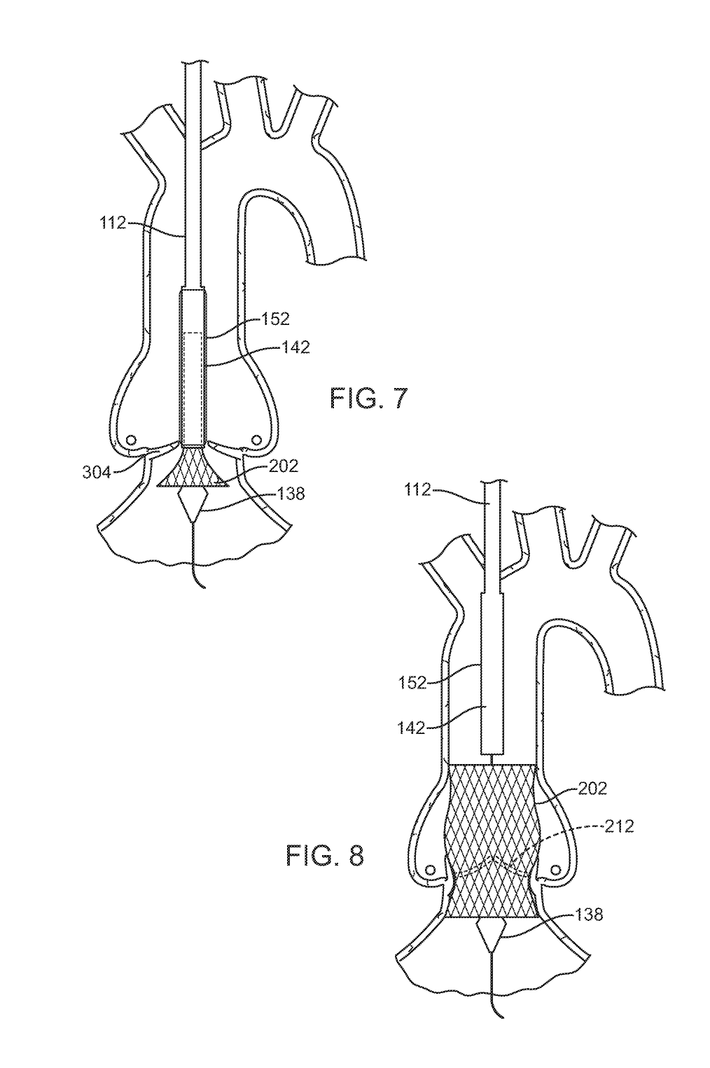

As shown in FIG. 7, after deflation of balloon 152, capsule 142 is withdrawn proximally, thereby permitting valve prosthesis 202 to self-expand.

As valve prosthesis 202 expands, it traps the leaflets of the patient's defective aortic valve against the valve annulus, retaining the native valve in a permanently open state. The outflow section of the valve prosthesis expands against and aligns the prosthesis within the ascending aorta, while the inflow section becomes anchored in the aortic annulus of the left ventricle, so that the valve prosthesis skirt reduces the risk of perivalvular leaks.

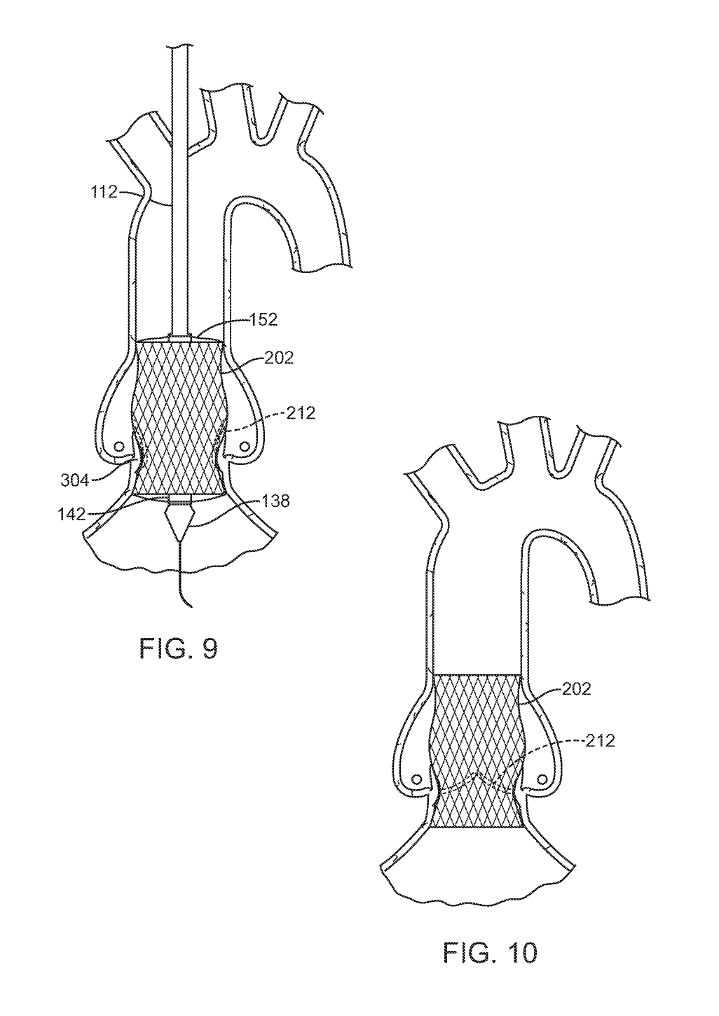

Referring now to FIG. 9, in certain cases, dilation of the prosthetic valve is required after valve delivery in order to properly seat the valve prosthesis, prevent leakage, and/or to remove residual calcification on the native valve. This post valve prosthesis delivery dilation procedure can also be performed using balloon 152 on integrated delivery system 10 after valve prosthesis 202 is delivered and expanded into aortic annulus 306. After deployment of valve prosthesis 202, tip 138 of integrated delivery system 10 is withdrawn proximally to abut the distal end of capsule 142. The integrated delivery system 10 is then advanced into valve prosthesis 202, across replacement valve 212. Once positioning of the integrated delivery system 10 is confirmed, post deployment balloon dilation is performed by inflating balloon 152 into valve prosthesis 202 and aortic annulus 306.

Alternatively, the integrated delivery system and valve prosthesis can be advanced through a transapical procedure. In a transapical procedure, a trocar or overtube is inserted into the left ventricle through an incision created in the apex of a patient's heart. A dilator is used to aid in the insertion of the trocar. In this approach, the native valve (e.g. the mitral valve) is approached from the downstream relative to the blood flow. The dilation balloon is attached to an exterior surface of a distal end of the trocar. Balloon dilation is performed by expanding the balloon into the native valve. Then the trocar is retracted sufficiently to release the self-expanding valve prosthesis. The dilator is preferably presented between the valve leaflets. The trocar can be rotated and adjusted as necessary to properly align the valve prosthesis. The dilator is advanced into the left atrium to begin disengaging the proximal section of the valve prosthesis from the dilator. In an alternate aspect of the invention, the integrated delivery system can function as a trocar, thus eliminating the need for an overtube or dilator. In this aspect, tip 138 functions as a trocar to penetrate the incision.

In an alternate aspect of the invention, the valve prosthesis can be delivered through a transatrial procedure. In this procedure, the dilator and trocar are inserted through an incision made in the wall of the left atrium of the heart. The dilator and trocar are advanced through the native valve and into the left ventricle of heart. The dilator is then withdrawn from the trocar. A guide wire is advanced through the trocar to the point where the valve prosthesis comes to the end of the trocar. Balloon dilation is performed by expanding the balloon into the native valve. Then the valve prosthesis is advanced sufficiently to release the self-expanding frame from the trocar. The trocar can be rotated and adjusted as necessary to properly align the valve prosthesis. The trocar is completely withdrawn from the heart such that the valve prosthesis self-expands into position and assumes the function of the native valve. In an alternate aspect of the invention, the integrated delivery system can function as a trocar, thus eliminating the need for an overtube or dilator. In this aspect, tip 138 functions as a trocar to penetrate the incision.

The foregoing description has been presented for purposes of illustration and enablement, and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

What is claimed is:

1. An integrated balloon dilation and valve prosthesis delivery device comprising:
an inner shaft assembly including an intermediate portion providing a coupling structure configured to selectively engage a prosthetic heart valve; and
an outer shaft assembly including:
a delivery sheath capsule at a distal end of the outer shaft assembly, the capsule being slidably disposed over the inner shaft assembly and configured to compressively contain a prosthetic heart valve engaged with the coupling structure,
an expandable balloon attached to an exterior surface of the capsule, and
an inflation lumen extending along the length of the outer shaft assembly, configured to transmit fluid into the balloon for expansion.

2. The integrated device of claim 1, wherein the inflation lumen extends along an interior of the outer shaft assembly.

3. The integrated device of claim 1, wherein the inflation lumen extends along an exterior of the outer shaft assembly.

4. The integrated device of claim 1, wherein the prosthetic heart valve is self-expanding.

5. The integrated device of claim 1, wherein the balloon is attached to the exterior surface of the capsule at a proximal end of the capsule and a distal end of the capsule.

6. The integrated device of claim 1, wherein the capsule is configured to compressively contain the prosthetic heart valve within an interior area of the capsule.

7. The integrated device of claim 1, wherein the inflation lumen is connected to the balloon at a location that is configured to be proximal to the coupling structure.

8. The integrated device of claim 1, wherein the inflation lumen is connected to the balloon at a proximal end of the balloon.

9. The integrated device of claim 1, wherein the prosthetic heart valve includes a frame and a plurality of valve leaflets, the frame being configured to engage the coupling structure.

10. A method of treating a valve disorder in a patient's heart, comprising:
delivering an integrated balloon dilation and valve prosthesis delivery device through an anatomic passageway into a native valve within a heart, the integrated device including a capsule and a balloon at a distal end, the capsule surrounding a collapsed valve prosthesis, the balloon being attached to an exterior surface of the capsule;
expanding the balloon to contact and dilate the native valve;
proximally retracting the capsule relative to the valve prosthesis to deploy the valve prosthesis in the native valve.

11. The method of claim 10, further comprising:
delivering the integrated device into the deployed valve prosthesis; and
expanding the balloon to contact and dilate the valve prosthesis.

12. The method of claim 10, further comprising contracting the balloon prior to proximally retracting the capsule.

13. The method of claim 10, wherein the valve prosthesis includes a valve body having a plurality of valve leaflets attached to a frame.

14. The method of claim 10, wherein the integrated device is delivered through the subclavian artery.

15. The method of claim 10, wherein the integrated device is delivered through the femoral artery.
